(19) United States
(12) Patent Application Publication
(10) Pub. No.: US 2012/0323216 A1
(43) Pub. Date: Dec. 20, 2012

(54) NEEDLESHIELD ASSEMBLY

(76) Inventor: LAWRENCE R. KOH, North Hollywood, CA (US)

(21) Appl. No.: 13/162,548

(22) Filed: Jun. 16, 2011

(51) Int. Cl. A61M 5/32; A61M 5/50

(52) U.S. Cl. 604/506; 604/263

(57) ABSTRACT

A needleshield assembly including a needle hub, hypodermic needle and protective cover is disclosed in which the protective cover can both secure and disable the needle following use.

### FIELD

The present application relates to hypodermic needles, and more particularly to disposable, non-reusable needles with shields.

### BACKGROUND

Hypodermic syringes are used with hypodermic needles to inject liquid into body tissues or to remove fluids or other samples, such as blood, from the body. The barrel of a syringe can be made of plastic or glass and usually has graduated marks indicating the volume of fluid in the syringe. The barrel of a syringe is nearly always transparent. Most modern medical syringes are plastic with a rubber piston because this type of syringe seals much better between the piston and the barrel and because they are cheap enough to dispose of after being used only once. Very serious problems, such as the transmission of disease-causing viruses, can arise with needle re-use, in particular outside of health care settings.

At least 3 billion injections occur yearly outside of health care settings. About 2 billion of these injections are administered by people with diabetes and patients receiving home health care. Approximately 1 billion are attributed to injection drug users (IDUs) using illicit drugs like heroin and cocaine. Most of the needles used for these injections end up discarded in household trash and community solid waste, putting workers and the public at risk of needle stick injuries. Problems that can arise from unsafely discarded used syringes and needles include needle stick injuries and potentially fatal blood borne infections, such as human immune deficiency virus (HIV) and hepatitis B and C. If a needle stick injury occurs, the costs of providing post-injury counseling and prevention measures are significant.

Even in a controlled setting such as a hospital or other healthcare facility, an accidental needle stick injury is a constant occupational hazard for healthcare workers and other employees working in such a setting. Although such facilities generally provide disposal containers (e.g., Covidien™ Sharps Containers), improper disposal of syringes and needles can increase the risk of an accidental needle stick injury, for example if a healthcare worker or employee is not be properly educated in the proper disposal of needles and inadvertently disposes of a used needle in the regular trash. A healthcare worker might also sustain an accidental needle stick in the course of administering medication to a patient.

### SUMMARY

In view of the risks of needle stick injuries, there remains a need for needle systems that reduce the risks of such injuries occurring. The present needleshield assembly meets this need by rendering a needle system non-usable after a single use.

The present needleshield assembly (200) comprises a needle assembly and a cover (220). The needle assembly includes a needle (210), a needle hub (202), and a coupling member (206) for retaining the needle hub (202). The needle (210) extends longitudinally from the distal end of the needle hub (202), and the needle hub (202) includes at least one locking protrusion (218) extending outwardly and laterally from the needle hub (202). The cover (220) is connected to the needle (210) of the needle assembly by a hinge (208).

The cover (220) of the present needleshield assembly (200) includes a hollow elongate body having a longitudinal aperture (228), the longitudinal aperture (228) being sized to allow passage of the needle (210) and needle hub (202) therethrough. The needle (210) and needle hub (202) can be placed into or removed from the elongate body of the cover (220) by rotating the needle assembly around the hinge (208) and passing the needle (210) and needle hub (202) through the longitudinal aperture (228). Also included in the cover (220) is at least one lock aperture (228) in the elongate body, for receiving locking protrusions (218) on the needle hub (202). In preferred embodiments, the needle hub (202) comprises two locking protrusions (218) on laterally opposite sides of the needle hub (202), and the cover (220) comprises two lock apertures (228) for receiving the respective locking protrusions (218), in order to more securely retain the needle assembly within the cover (220) following use and discourage re-use of the needle assembly. The cover (220) further includes an inwardly projecting protrusion (224) for puncturing and thereby disabling the needle assembly following use of the present system. The protrusion (224) preferably has a sharp edge at a distal end and is situated within the proximal end of the elongate body of the cover (220).

In order to disable the needle assembly of the present system, the needle assembly is rotated around the hinge (208) from an open configuration in which the needle (210) and needle hub (202) are outside the cover (220) to a fully closed configuration in which the needle (210) and needle hub (202) are retained within the cover (220) and the locking protrusion (218) of the needle hub (202) is received within the lock aperture (228) of the cover (220). When this occurs, the inwardly projecting protrusion (224) of the cover (220) is simultaneously brought into contact with an outer surface of the needle hub (202) so as to pierce the surface of the needle hub (202). The needle assembly is thereby secured within the cover (220) and rendered incapable of reuse when placed in the fully closed configuration.

In preferred embodiments, the present needleshield assembly (200) further comprises at least one retaining pin (214) extending outwardly and laterally from the needle hub (202), the retaining pin (214) being located distally with respect to the locking protrusion (218) and being sized and positioned to be received within the lock aperture (228) of the elongate body of the cover (220). The retaining pin (214) maintains the needle assembly within the cover (220) in a configuration ready for use, thereby preventing injury to a user prior to use and preferably also maintaining sterility. Preferably, the needle hub (202) comprises two retaining pins on laterally opposite sides of the needle hub (202), each retaining pin (214) being sized and positioned to be received within a corresponding lock aperture (228) in the elongate body of the cover (220).

The coupling member (206) of the needle assembly preferably comprises a distal end that extends laterally away from the needle hub (202) and forms a flange. The flange facilitates the opening and closing of the present needleshield assembly (200) through the application of pressure to the flange. The coupling member (206) also preferably comprises a proximal end having a lower surface which is adapted to contact and preferably mate with an upper surface of the cover (220) adjacent the hinge (208) when the needle assembly is rotated into the fully closed configuration. Preferably, the lower surface of the coupling member (206) and the upper surface of the cover (220) are substantially planar.

The cover (220) of the needleshield assembly (200) also preferably comprises a lever (230) protruding from an exterior surface of the proximal end of the cover (220), to facilitate rotation of the cover (220) around the hinge (208). The lever (230) is attached at a proximal end of the cover (220), and in a preferred embodiment extends distally in a curved manner.

In addition, the elongate body of the cover (220) preferably comprises a pair of parallel, longitudinally extending walls connected along one longitudinal end by a connecting wall opposite the longitudinal aperture (228). When the present needleshield assembly (200) is placed in the fully closed configuration, the distal end of the needle (210) preferably contacts the connecting wall and the needle (210) is bent, thereby rendering it unfit for use by a subject. The longitudinally extending walls can also further comprise a flange (216) extending from an interior surface in a direction inwardly and away from the longitudinal aperture (228) for retaining the needle (210) following use. Prior to use, the needle (210) is located medially with respect to the flange (216), but following use the needle (210) passes over the flange (216) when the needleshield assembly (200) is closed and becomes situated laterally when the needle assembly has been placed in the fully closed configuration. The needle (210) is then restrained by the flange (216) from being passed through the longitudinal aperture (228).

In use, the present needleshield assembly (200) can be used to dispense or withdraw a fluid with a needle (210) and to prevent reuse of the needle (210). When the present needleshield assembly (200) is placed in an open configuration such that the needle (210) and needle hub (202) are outside the cover (220), the needle hub (202) can be attached, for example, to a syringe, and a liquid can then be dispensed or withdrawn through the needle (210) and needle hub (202), such as in connection with an injection of a medicament. Following such use, the needleshield assembly (200) can be rotated around the hinge (208) from the open configuration to a fully closed configuration in which the needle (210) and needle hub (202) are retained within the cover (220) and the locking protrusion (218) of the needle hub (202) is received within the lock aperture (228) of the cover (220). The inwardly projecting protrusion (224) is simultaneously brought into contact with the outer surface of the needle hub (202) so as to pierce the surface of the needle hub (202) and render it incapable of reuse. Preferably, the present needleshield assembly (200) is first placed in a closed, ready-to-use configuration by placing the needle and needle hub (202) within elongate body of the cover (220) until the retaining pin (214) is retained within the lock aperture (228) of the cover (220). In order place the needleshield assembly (200) in the fully closed configuration, pressure can be applied to a lever protruding from an exterior surface of the cover (220).

### DRAWINGS

The features, nature, and advantages of the present invention will become more apparent from the detailed description set forth below when taken in conjunction with the drawings, in which like reference characters identify corresponding features.

FIG. 1 illustrates a prior art syringe and hypodermic needle with attached Luer lock.

FIG. 2 is a side elevation view of an embodiment of the present needleshield assembly in a closed, unused configuration.

FIG. 3 is a perspective view of the embodiment shown in FIG. 2.

FIG. 4 is a plan view of the needleshield assembly of FIG. 2 taken from the distal end of the cover.

FIG. 5 is a side elevation view of the needleshield assembly in of FIG. 2 in a partially open configuration.

FIG. 6 is a perspective view of the needleshield assembly of FIG. 2 in an open, ready-to-use configuration.

FIG. 7 is another perspective view of the needleshield assembly of FIG. 2 in an open, ready-to-use configuration.

FIG. 8 is yet another perspective view of the needleshield assembly of FIG. 2 in an open, ready-to-use configuration.

FIG. 9 is a plan view of the needleshield assembly of FIG. 2 in an open, ready-to-use configuration, as viewed from the proximal end of the cover.

FIG. 10 is a side elevation view of the needleshield assembly of FIG. 2 in a fully closed, used configuration.

FIG. 11 is an enlarged view of a portion of the side elevation view of FIG. 10, showing the puncturing protrusion engaged with the sidewall of the needle hub of the present needleshield assembly in a fully closed, used configuration.

FIG. 12 a top plan view of the needleshield assembly of FIG. 2 in a closed configuration following use of the present device and prior to puncture, as viewed from the distal end of the cover.

FIG. 13 is a plan view of needleshield assembly of FIG. 2 prior to use, attached to a syringe and provided in a package.

### DESCRIPTION

Definitions

As used herein, the following terms and variations thereof have the meanings given below, unless a different meaning is clearly intended by the context in which such term is used.

“Hollow,” with respect to a component of the present device, refers to the presence of an empty space between walls of the component which is not otherwise filled by a part of the component.

“Hypodermic needle” means an implement for discharging liquids into and/or withdrawing liquids from a container and/or from the tissue of a subject. Hypodermic needles typically comprise a needle hub and a needle.

“Inward” and “inwardly” refer to a direction toward the interior of a component of the present device.

“Lateral” refers to a direction away from a longitudinal axis of a component of the present device.

“Longitudinal” refers to the direction of the longest axis of a component of the present device.

“Medial” refers to a direction toward a longitudinal axis of a component of the present device.

“Needle” refers to the hollow shaft portion (cannula) of a hypodermic needle having a tip at a distal end, typically made from metal.

“Needle hub” or “hub” refers to the portion of a hypodermic needle which is connected at a distal end to the shaft portion of a needle and which comprises at a proximal end an opening for connecting to a mating portion of a syringe, bottle, or other container for a liquid.

“Outward” and “outwardly” refer to a direction toward the exterior of a component of the present device.

“Sharp” as pertaining to a sharp edge refers to an edge which is substantially non-radiused to facilitate cutting or piercing a surface.

“Subject” refers to a mammal, preferably a human, though in some embodiments a subject can also be an animal, e.g., a companion animal (e.g., dogs, cats, and the like), farm animal (e.g., cows, sheep, pigs, horses, and the like) or laboratory animal (e.g., rats, mice, guinea pigs, and the like) in connection with veterinary treatment or experimentation.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Needleshield Assembly

The following detailed description is of the best currently contemplated modes of carrying out the present application. The description is not to be taken in a limiting sense, but is made for purpose of illustrating the general principles of the present invention.

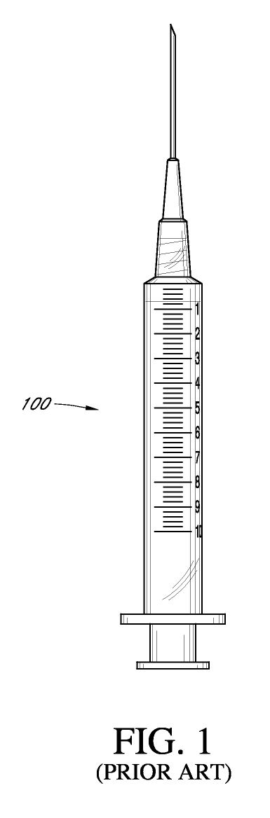

FIG. 1 illustrates a prior art syringe and hypodermic needle 100 with attached Luer lock. As described above, the sharp point of the needle is exposed following use, and may be contaminated with a virus or other communicable agent.

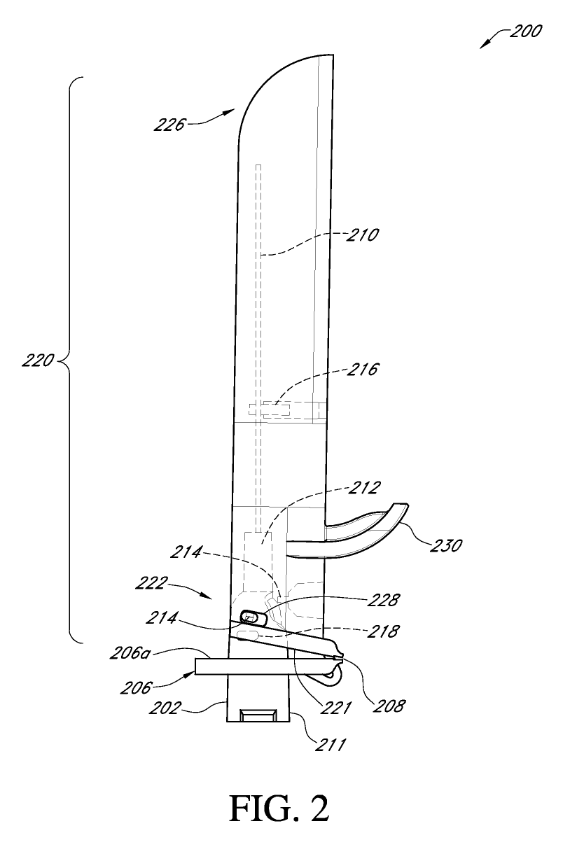

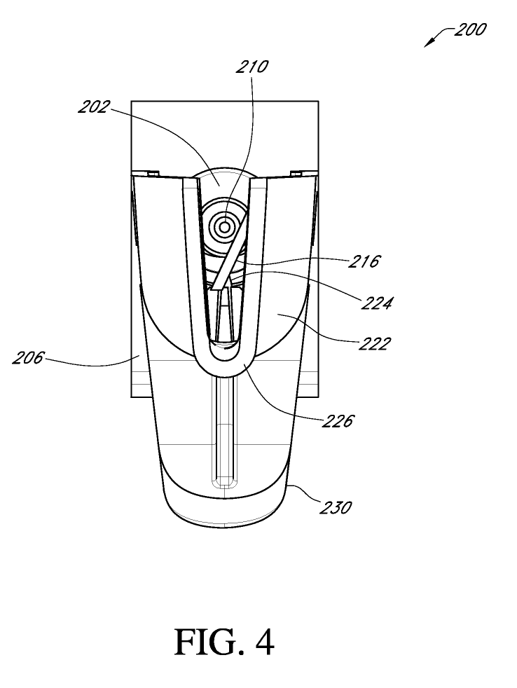

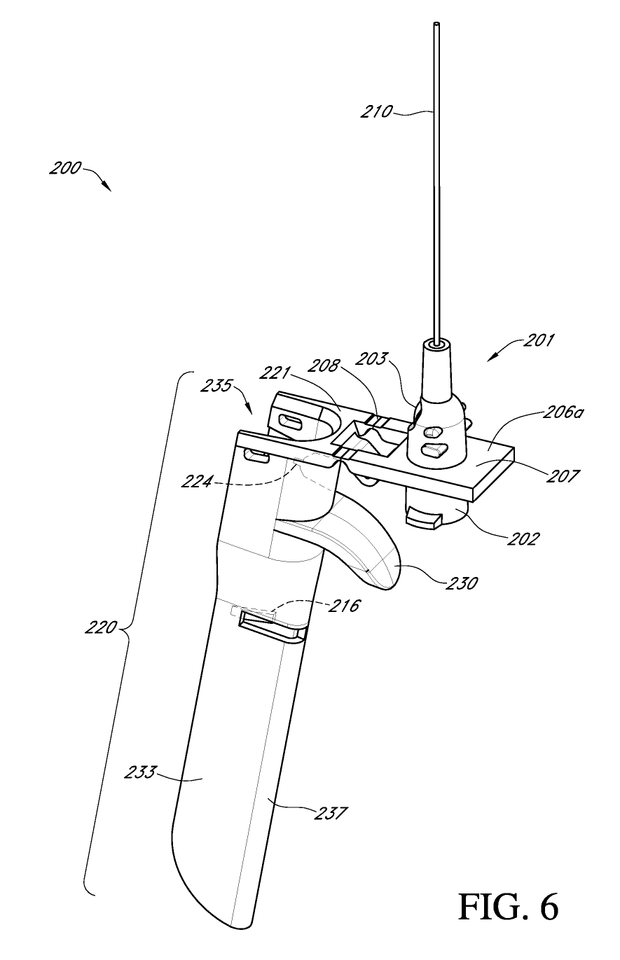

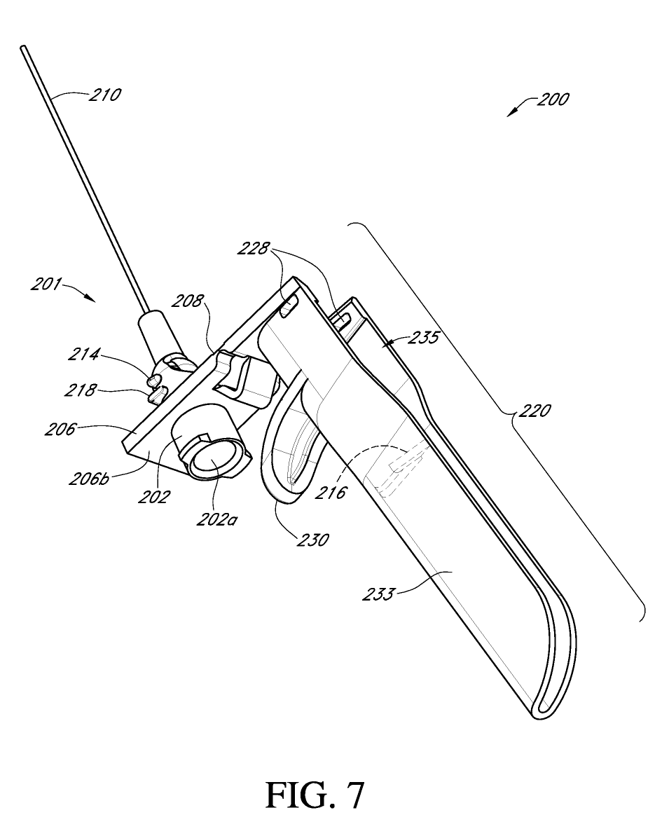

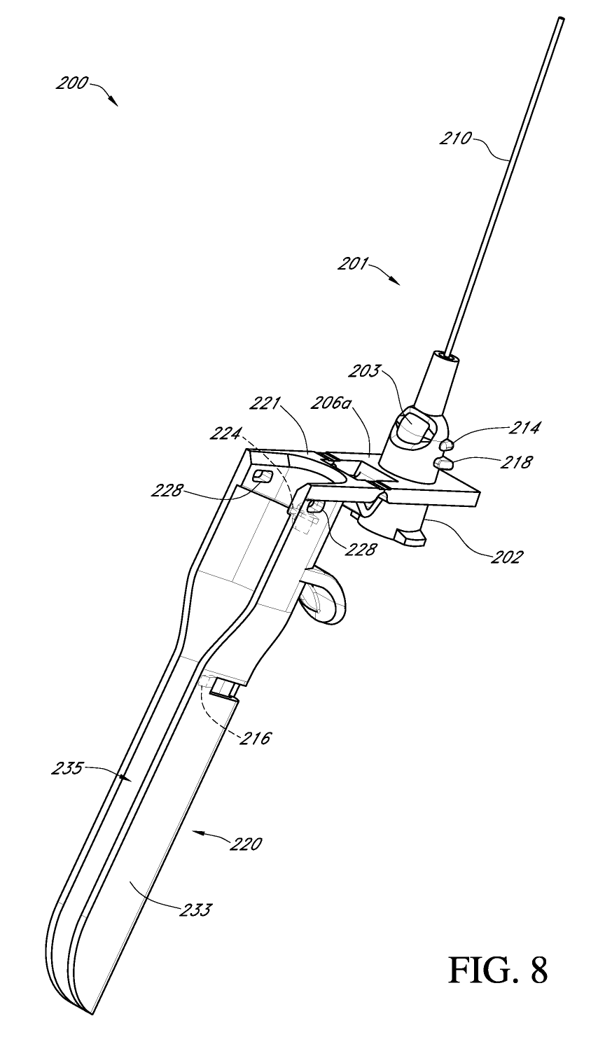

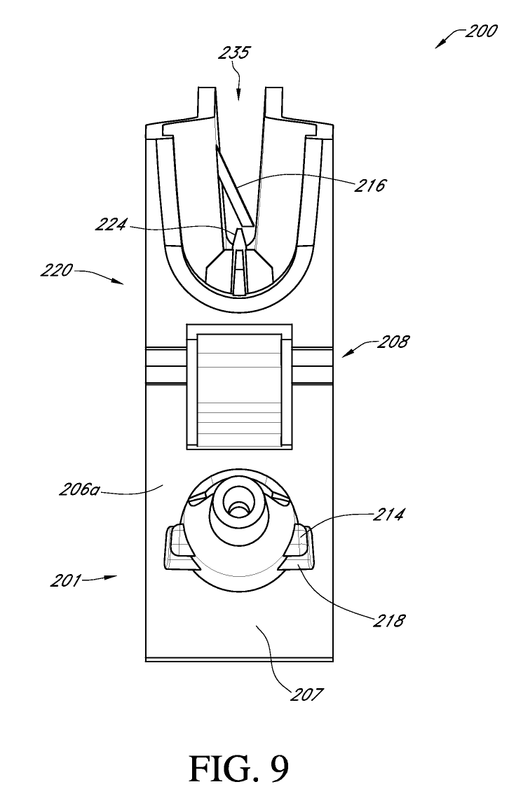

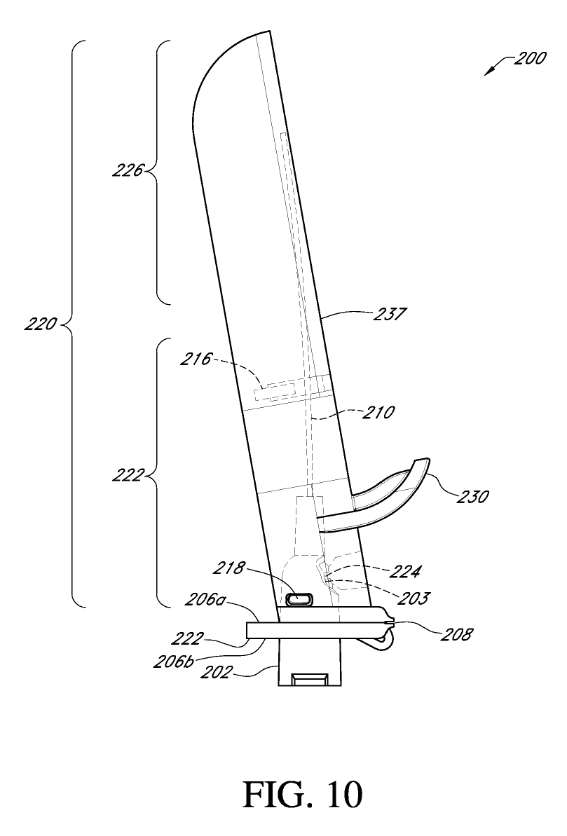

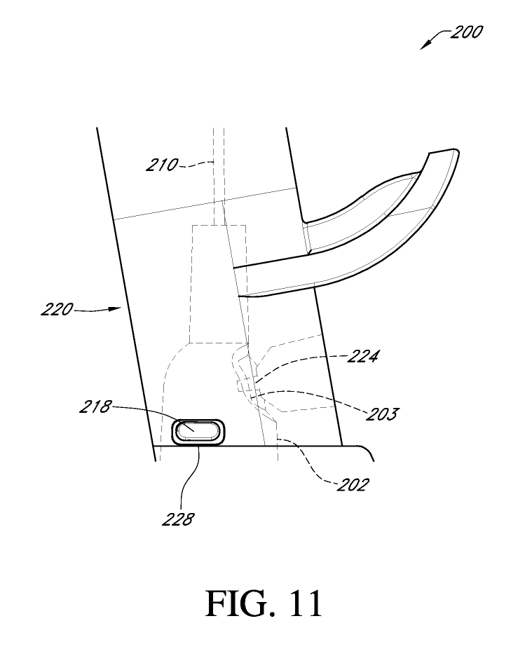

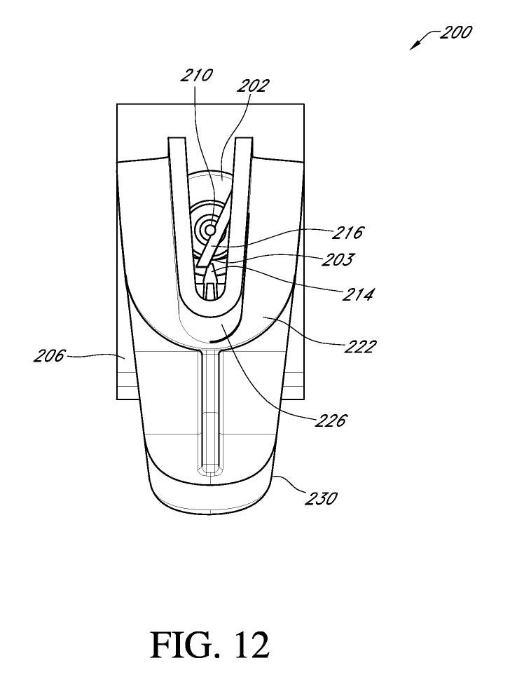

The present needleshield assembly 200 can advantageously assume one of three general configurations: a closed, unused configuration (shown in FIGS. 2-4 and 13), an open configuration (as shown in FIGS. 6-9) and a fully closed configuration (shown in FIGS. 10-12). In the closed, unused configuration, the needle 210 of the needleshield assembly 200 is covered by a cover 220, thereby preventing injury to a user of the present needleshield assembly 200 prior to use and facilitating manufacture of the needleshield assembly by avoiding the need for a separate needle cover. Once opened, the needleshield assembly 200 can be attached to a syringe in order to withdraw fluids and/or perform an injection. Following such use, the present needleshield assembly 200 is then preferably placed in the fully closed configuration, thereby rendering it unfit for re-use, as described further below.

FIG. 2 illustrates an embodiment of the present needleshield assembly 200, which includes a needle assembly 201 and a cover 220 that can function both to protect a needle 210 and needle hub 202 prior to use and to cover the needle 210 following use in order to prevent needlestick injuries. The needle assembly 201 includes a needle hub 202, a coupling member 206 for retaining the needle hub 202, and, optionally, a needle 210. The needle 210 extends longitudinally from the distal end of the needle hub 202, and the needle hub 202 includes at least one locking protrusion 218 extending outwardly and laterally from the needle hub 202. The needle hub 202, coupling member 206, and cover 220 are advantageously made from a medical grade, rigid plastic such as polypropylene, while the needle 210 can be made from steel or other materials used in making medical cannulas.

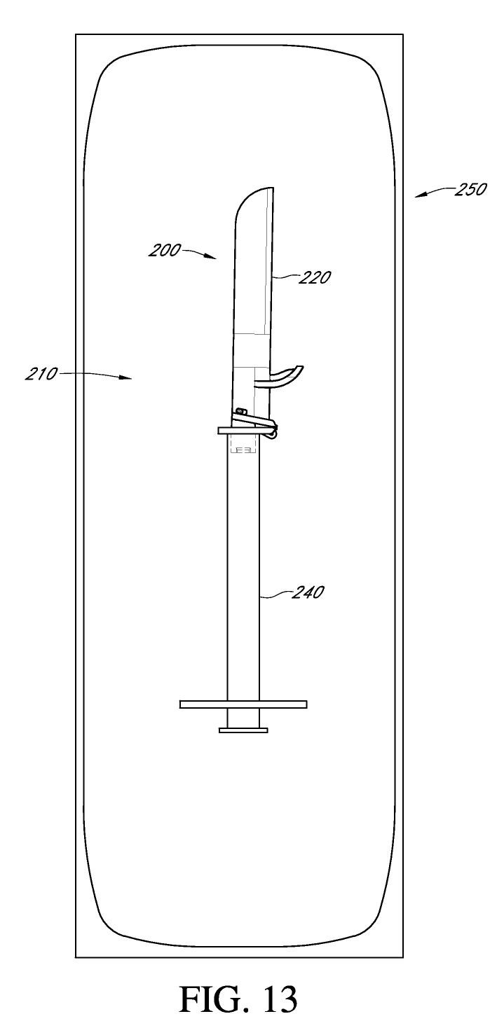

The needle hub 202 is configured to couple the needleshield assembly 200 to a fluid delivery device, as seen for example in FIG. 13, via a proximal opening 202a which provides fluid communication between the fluid delivery device and the needle assembly 201. The proximal opening 202a is also preferably adapted to provide a secure connection between the needle hub 202 and a fluid delivery device, which can be, for example, a syringe, a catheter assembly, a blood transfusion connection, or a peripheral device. The proximal opening 202a can preferably be configured to attach to standard syringes, such as via a Luer (6%) taper connection, e.g. a LUER-LOK or LUER SLIP connection, or via other suitable attachment mechanisms.

In preferred embodiments, the needle hub 202 comprises two locking protrusions 218 on laterally opposite sides of the needle hub 202 which extend outwardly and laterally from the needle hub 202, and the cover 220 comprises two lock apertures 228 for receiving the respective locking protrusions 218. When the present needleshield assembly 200 is in the fully closed configuration, the locking protrusions 218 are retained within the lock apertures 228, and this mechanism operates to securely retain the needle assembly 201 within the cover 220 following use of the needle assembly 201 and to discourage re-use of the needle assembly 201.

In the closed, unused configuration, the set of locking protrusions 218 do not engage with the corresponding lock apertures 228 of the cover 220. Instead, in preferred embodiments, the present needleshield assembly 200 further comprises at least one retaining pin 214 extending outwardly and laterally from the needle hub 202, the retaining pin 214 being located distally on the needle hub 202 with respect to the locking protrusion 218 and being sized and positioned to be received within the lock aperture 228 of the elongate body of the cover 220. The retaining pin 214 maintains the needle assembly 201 within the cover 220 in a configuration ready for use when it is retained in a lock aperture 228, thereby preventing injury to a user prior to use and preferably also maintaining sterility. Preferably, the needle hub 202 comprises two retaining pins on laterally opposite sides of the needle hub 202, each retaining pin 214 being sized and positioned to be received within a corresponding lock aperture 228 in the elongate body of the cover 220.

The needle hub 202 is retained by coupling member 206. In a preferred embodiment he coupling member can be approximately rectangular in configuration, though other suitable geometric configurations are within the scope of the invention. The coupling member 206 is preferably integrally molded or otherwise formed with the needle hub 202. Alternatively, the coupling member 206 can be formed with an aperture therethrough which allows the coupling member 206 to be fixedly coupled to a barrel portion of the needle hub 202 such that the needle hub 202 is securely situated within the aperture of the coupling member 206.

The coupling member 206 is preferably joined to the protective cover 220 by a hinge 208 which allows rotation of the needle assembly 201 relative to the cover 220. In an embodiment in which the coupling member 206 is approximately rectangular in configuration, the hinge 208 can be located at one lateral edge of the coupling member 206. The coupling member 206 of the needle assembly 201 preferably comprises a distal end that extends laterally away from the needle hub 202 (and away from the hinge 208) and forms a flange 207. The flange 207 facilitates the opening and closing of the present needleshield assembly 200 as it allows a user to apply pressure to the flange 207 and thereby obtain leverage when opening or closing the present needleshield assembly 200.

The coupling member 206 also preferably comprises a proximal end adjacent the hinge 208 having a distal surface 206a which is adapted to contact and preferably mate with a proximal surface 221 of the cover 220 when the needle assembly 201 is rotated into the fully closed configuration. By mating or being positioned in close proximity to one another, the proximal surface 221 and distal surface 206a make it difficult for a potential user to pry open the fully closed needleshield assembly 200 and thereby discourage or prevent re-use of the needle assembly 201. Preferably, the distal surface 206a of the coupling member 206 and the proximal surface 221 of the cover 220 are substantially planar.

In one embodiment, the protective cover 220 is sized to partially, substantially or completely encapsulate the needle 210 and needle hub 202. The cover 220 of the present needleshield assembly 200 includes a hollow elongate body having a longitudinal aperture 235, the longitudinal aperture 235 being sized to allow passage of the needle 210 and needle hub 202 therethrough. The needle 210 and needle hub 202 can be placed into or removed from the elongate body of the cover 220 by rotating the needle assembly 201 around the hinge 208 and passing the needle 210 and needle hub 202 through the longitudinal aperture 235. Also included in the cover 220 is at least one lock aperture 228 in the proximal end 222 of the elongate body, for receiving locking protrusions 218 and/or retaining pins 214 on the needle hub 202.

The elongate body of the cover 220 preferably comprises a pair of longitudinally extending walls 233, each connected along one longitudinal end by a connecting wall 237 positioned opposite the longitudinal aperture 235. The longitudinally extending walls 233 can be substantially planar and parallel along some or all of their extent. Preferably, the interior surfaces of each of the elongate walls 233 of the cover 220 are proximate to the needle 210 and to the needle hub 202 along the majority of the length of the needle assembly 201, or along the entire length of the needle assembly 201, in order to make it difficult for a potential user to pry the needle assembly 201 out of the cover 220 when the needle assembly 200 is in the fully closed configuration. When the present needleshield assembly 200 is placed in the fully closed configuration, the distal end of the needle 210 preferably contacts the connecting wall 237 and the needle 210 is bent, thereby rendering it unfit for use by a subject.

In one embodiment, from a proximal end 222 to a distal end 226 thereof, the protective cover 220 can approximate a cuboid configuration terminating in a curved distal end 226. The protective cover 220 can for example have a three-sided cuboid configuration wherein the three sides (i.e., first side, back side, and second side) represent lengths of the cuboid. The fourth “side” comprises the opening 235. In one embodiment, when moving from the proximal end 222 to the distal end 226, there is a narrowing of the distance between the first side and second side, i.e. between the two elongate walls 233, in order to remain proximate the exterior surfaces of the needle 210 and the needle hub 202 when the needle assembly 201 is within the cover 220.

The cover 220 further includes an inwardly projecting protrusion 224 protruding from an interior surface of the cover 220 for puncturing and thereby disabling a surface 203 of the needle hub 202 following use of the present needleshield assembly 200. The protrusion 224 preferably has a sharp edge at a distal end and is situated within the proximal end 222 of the elongate body of the cover 220. In the closed, unused configuration, the protrusion 224 of the needleshield assembly 200 can be in close proximity to a portion of the wall of the needle hub 202, but preferably does not contact such surface. FIGS. 2-4 illustrates the puncturing protrusion 224 in close proximity to portion 203 of the wall of the needle hub 202, with the needleshield assembly 200 being in the closed, unused configuration.

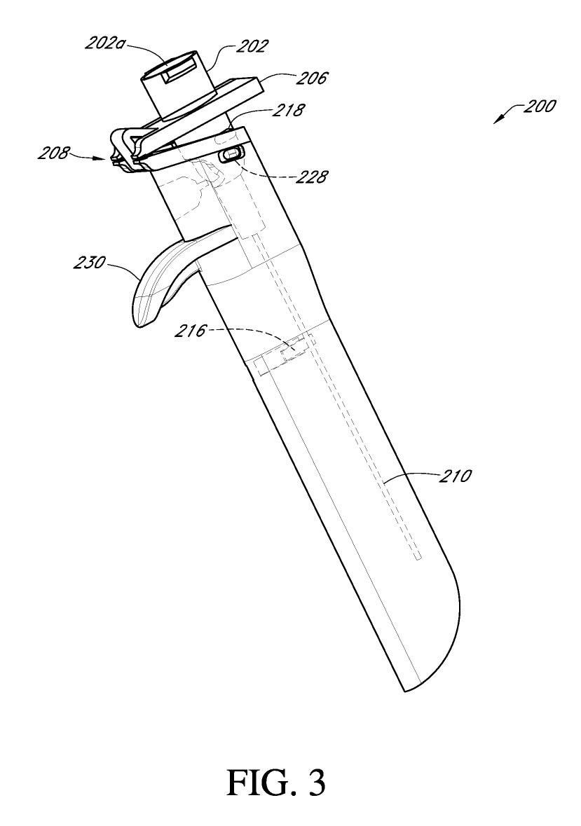

The protective cover 220 can also include a hooking mechanism (flange 216) protruding from an interior surface thereof. Relative to the needle 210, the hooking mechanism is preferably positioned below the most distal point of the needle 210. The flange 216 extends from an interior surface of the longitudinally extending walls 233 or 237 of the cover 220 in a direction inwardly and away from the longitudinal aperture 235 in order to retain the needle 210 following use. Prior to use, the needle 210 is located medially with respect to the flange 216, but following use, when the needleshield assembly 200 is placed in the fully closed configuration, the needle 210 passes over the flange 216 when the needleshield assembly 200 is closed and becomes situated laterally of the flange 216 when the needle assembly 201 has been placed in the fully closed configuration. The needle 210 is then restrained by the flange 216 from being passed through the longitudinal aperture 235. In the closed, unused configuration, the hooking mechanism 216 of the needleshield assembly 200 may not necessarily contact the needle 210. FIG. 3 illustrates the flange 216 of the hooking mechanism protruding from an interior surface of the protective cover 220 and not in contact with the needle needle 210.

FIG. 10 illustrates a further safety feature of the present needleshield assembly, namely the bending of the needle 210 when the assembly 200 is placed in the fully closed configuration, thereby rendering the needle 210 unfit for further use. This is accomplished by providing a proximal surface 221 of the cover 220 which extends from the hinge 208 at an acute angle (i.e., less than 90 degrees) with respect to the connecting or rear wall 237 opposite the cover opening or longitudinal aperture 235 of the protective cover 220. When a distal surface 206a of the coupling member 206 is provided which is approximately perpendicular to the needle 210, and when the distal surface 206a of the coupling member 206 meets or comes in close proximity to the proximal surface 221 of the cover 220, the result is that the needle 210 and connecting wall 237 are non-parallel, and a distal portion of the needle 210 therefore contacts the connecting wall surface and is bent by it.

The cover 220 of the needleshield assembly 200 also preferably comprises a lever 230 protruding from an exterior surface of the proximal end of the cover 220, to facilitate rotation of the cover 220 around the hinge 208. The lever 230 is attached at a proximal end of the cover 220, and in a preferred embodiment extends distally in a curved manner. Such an arched lever protrusion 230 protrudes from an exterior surface of the protective cover 220, with the arched lever adapted to direct the protective cover from the closed position to the open position, and vice-versa. The arched lever 230 can be approximately sized to accommodate a thumb or finger of a user. Alternatively, the protective cover can comprise a defined indentation along the exterior surface at the proximal end of the cover 220 in order to direct the protective cover 220 from a closed position to an open position, and vice-versa.

FIGS. 6-8 illustrate perspective views of a needleshield assembly 200 in an open, ready-to-use configuration according to an embodiment of the invention. In this embodiment, the protective cover 220 completely exposes the needle 210. This can be accomplished by a user when the user rotates the needle assembly 201 around the hinge 208 and opens the protective cover 220 from the closed, unused configuration (see FIG. 2), thereby exposing the needle 210 such that the needleshield assembly 200 is in the open, ready-to-use configuration. The open fourth “side” of the protective cover 220 allows the protective cover 220 to move from the closed configuration to the open configuration, and vice-versa, without contacting the needle 210.

As shown, the hinge 208 keeps the protective cover 220 connected to the coupling member 206, remains securely coupled to the barrel of the needle hub 202. The hinge 208 is thus capable of keeping the protective cover 220 in a closed configuration (shielding the needle cannula, as in FIGS. 2-4 and 10-12) or in an open, ready-to-use configuration (exposing the needle cannula for use). In the open, ready-to-use configuration, the needle 210 of the needleshield assembly 200 can be used to dispense, i.e., administer, medication to a patient. After administration of medication, the protective cover 220 of the needleshield assembly 200 can be placed in a fully closed configuration to permanently and irreversibly cover, i.e., encapsulate, the used needle 210. When the needle assembly 201 is placed within the cover 220 in the fully closed configuration, the locking protrusions 218 preferably produce an audible and kinesthetic sensation (e.g., a clicking sound) as they pass into the lock apertures 228, thereby providing a user with an indication that the needleshield assembly 200 has been rendered inoperable.

### Method of Use

In use, the present needleshield assembly 200 can be used to dispense or withdraw a fluid with a needle 210 and to prevent reuse of the needle 210. Preferably, the needleshield assembly 200 is packaged in a manner that allows it to be kept clean and sterile until ready for use. An example of such packaging is shown in FIG. 13, in which the needleshield assembly 200 is individually packaged in sterile packaging 250 comprising a peel-off layer 210 that exposes a pre-assembled injection assembly comprising the present needleshield assembly 200 and a pre-attached syringe 240.

Whether provided with a pre-attached syringe 240 or not, the present needleshield assembly 200 is preferably manufactured for shipping and/or provided prior to use by a user in a closed, ready-to-use configuration, as shown in FIGS. 2, 3, and 13. In this configuration, the retaining pins 214 are received within the lock apertures 228, thereby securing and stabilizing the needleshield assembly 200 in the closed, ready-to-use configuration and preventing inadvertent opening of the needleshield assembly 200. As can be seen in FIGS. 2 and 3, in this configuration distal surface 206a of the coupling member 206 is positioned adjacent to proximal surface 221 of the cover 220, but is not in contact with the proximal surface 221. The present needleshield assembly 200 is preferably first placed in a closed, ready-to-use configuration by placing the needle and needle hub 202 within elongate body of the cover 220 until the retaining pin 214 is retained within the lock aperture 228 of the cover 220.

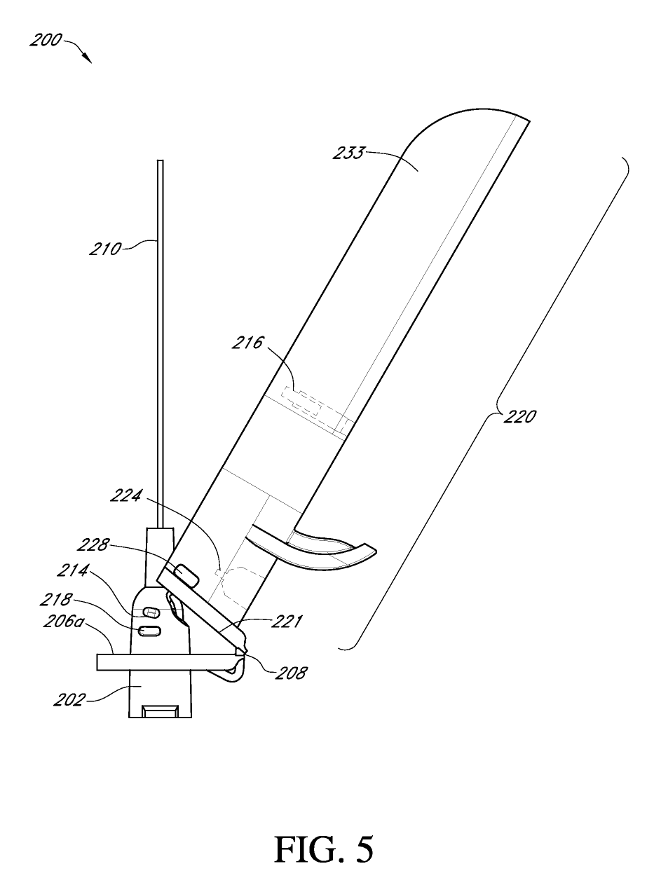

The present needleshield assembly 200 can then be placed in an open, ready-to-use configuration such that the needle 210 and needle hub 202 are outside the cover 220 by applying pressure to the distal surface 206a of the flange 207 coupling member 206, to the lever 230, and/or to the walls of the cover 220 so as to rotate the cover 220 and needle assembly 201 around the hinge 208, such that the proximal surface 221 of the cover 220 moves further from the distal surface 206a of the coupling member 206. Simultaneously, the retaining pins 214 are moved past the edges of the lock apertures 228 until they are no longer within the lock apertures 228 but instead are in contact with an interior wall of the cover 220. The retaining pins are preferably rounded (radiused) to facilitate such movement. As shown in FIGS. 5-8, by continuing to rotate the cover 220 and needle assembly 201 around the hinge 208, the retaining pins 214 are eventually moved so as to be no longer in contact with or be within the cover 220, and the needle hub 202 and needle 210 are also thereby passed through the opening 235 between longitudinally extending walls 233 of the cover 220.

When the present needleshield assembly 200 is in the fully open configuration, as shown in FIGS. 6-9, the needle hub 202 can be attached, for example, to a syringe, and a medicament, vaccine, or other liquid can then be dispensed or withdrawn through the needle 210 and needle hub 202. Following use of the needleshield assembly 200, and in particular following the insertion of the needle 210 into a tissue of a subject, such as in connection with an injection (intravenous, subcutaneous, or otherwise) and subsequent withdrawal of the needle 210, the needleshield assembly 200 can be rotated around the hinge 208 from the open configuration to a fully closed configuration in which the needle 210 and needle hub 202 are retained within the cover 220 and the locking protrusion 218 of the needle hub 202 is received within the lock aperture 228 of the cover 220. When this is accomplished, the inwardly projecting protrusion 224 is simultaneously brought into contact with the outer surface 203 of the needle hub 202 so as to pierce surface 203 of the needle hub 202 and render the needle assembly 201 incapable of reuse. Preferably, the protrusion 224 pierces a hole in the needle hub 202 that extends into the fluid passageway of the needle hub 202, although in some embodiments the protrusion 224 can operate only to secure the needle hub 202 to the cover 220. In order place the needleshield assembly 200 in the fully closed configuration, pressure can be applied to the lever 230 protruding from an exterior surface of the cover 220, to the flange 207, and or to the exterior of the cover 220.

FIG. 10 illustrates a side view of a needleshield assembly 200 in a closed, used configuration in which the needleshield assembly 200 has been disabled. In order to disable the needle assembly 201 of the present system, the needle assembly 201 is rotated around the hinge 208 from an open configuration in which the needle 210 and needle hub 202 are outside the cover 220 to a fully closed configuration in which the needle 210 and needle hub 202 are retained within the cover 220 and the locking protrusion 218 of the needle hub 202 is received within the lock aperture 228 of the cover 220. When this occurs, the inwardly projecting protrusion 224 of the cover 220 is simultaneously brought into contact with an outer surface of the needle hub 202 so as to pierce the surface of the needle hub 202. In one embodiment, the puncturing protrusion 224 irreversibly couples the protective cover 220 to the needle hub 202 when it punctures the outer surface of the needle hub 202, thereby preventing re-use. At the same time, as illustrated in FIG. 10, the needle 210 is bent as its distal end contacts the interior surface of rear wall 237 of the cover 220.

Thus, the present needleshield assembly 200 provides numerous mechanisms to prevent a needle from being used again after closing the assembly in the manner described previously. First, applying pressure to the indentation of the protective cover causes the puncturing protrusion to puncture the needle hub which irreversibly couples the protective cover to the needle hub. Second, the hook functions to secure the needle and keep it immobile once the protective cover is closed. Third, the flanges engage underneath the coupling mechanisms to extends provide further resistance to re-opening the protective cover after it has been used As a result, these mechanisms can either singly or combined function to prevent re-use of the needle after use thereof. Additionally, for manufacturing purposes, the needleshield assembly can be manufactured as one solid body sub-divided into several portions by hinges thereby realizing cost savings.

While certain exemplary embodiments have been described and shown in the accompanying drawings, it is to be understood that such embodiments are merely illustrative of and not restrictive on the broad application, and that this application is not limited to the specific constructions and arrangements shown and described, since various other modifications may occur to those ordinarily skilled in the art.

What is claimed is:

1. A needleshield assembly, comprising:
(a) a needle assembly comprising:
a needle;
a needle hub having a proximal end and a distal end, the needle extending longitudinally from the distal end of the needle hub, wherein the needle hub comprises a least one locking protrusion extending outwardly and laterally therefrom; and
a coupling member for retaining the needle hub;
(b) a cover connected to the coupling member of the needle assembly by a hinge, the cover comprising:
a hollow elongate body having a proximal end and a distal end and defining a longitudinal aperture, the longitudinal aperture sized to allow passage of the needle and needle hub therethrough, wherein the needle and needle hub are placed into the elongate body of the cover by rotating the needle assembly around the hinge and passing the needle and needle hub through the longitudinal aperture;
a lock aperture in the elongate body for receiving the locking protrusion; and
an inwardly projecting protrusion within the proximal end of the elongate body,
wherein the needle assembly can be rotated around the hinge from an open configuration in which the needle and needle hub are outside the cover to a fully closed configuration in which the needle and needle hub are retained within the cover and the locking protrusion of the needle hub is received within the lock aperture of the cover, wherein the inwardly projecting protrusion is simultaneously brought into contact with an outer surface of the needle hub so as to pierce the surface of the needle hub when the needle assembly is rotated into the fully closed configuration, the needle assembly being secured within the cover and rendered incapable of reuse when placed in the fully closed configuration.

2. The needleshield assembly of claim 1, wherein the needle hub comprises two locking protrusions on laterally opposite sides of the needle hub, and wherein the cover comprises two lock apertures for receiving the respective locking protrusions.

3. The needleshield assembly of claim 1, further comprising at least one retaining pin extending outwardly and laterally from the needle hub, the retaining pin being located distally with respect to the locking protrusion, wherein the retaining pin is sized and positioned to be received within the lock aperture of the elongate body of the cover and thereby maintain the needle assembly within the cover in a configuration ready for use.

4. The needleshield assembly of claim 3, wherein the needle hub comprises two retaining pins on laterally opposite sides of the needle hub, each retaining pin being sized and positioned to be received within a corresponding lock aperture in the elongate body of the cover.

5. The needleshield assembly of claim 1, wherein the elongate body of the cover comprises a pair of parallel longitudinally extending walls connected along one longitudinal end by a connecting wall opposite the longitudinal aperture.

6. The needleshield assembly of claim 5, wherein a proximal surface of the cover is disposed at an acute angle with respect to an interior surface of the connecting wall, and wherein a distal surface of the coupling member is disposed approximately perpendicular to the needle, such that when the needleshield assembly is placed in the fully closed configuration a distal end of the needle contacts the interior surface of the connecting wall, thereby bending the needle.

7. The needleshield assembly of claim 1, further comprising a flange extending from an interior surface of the elongate body in a direction inwardly and away from the longitudinal aperture, wherein the needle is located medially with respect to the flange in a first configuration of the needleshield assembly prior to use and laterally when the needle assembly has been placed in the fully closed configuration, thereby restraining the needle from being passed through the longitudinal aperture when the needle assembly has been placed in the fully closed configuration.

8. The needleshield assembly of claim 1, further comprising a lever protruding from an exterior surface of the proximal end of the cover to facilitate rotation of the cover around the hinge.

9. The needleshield assembly of claim 8, wherein the lever is attached at a proximal end to the cover and extends distally in a curved manner.

10. The needleshield assembly of claim 1, wherein the inwardly projecting protrusion has a sharp edge at a distal end.

11. The needleshield assembly of claim 1, wherein the coupling member comprises a distal end extending laterally away from the needle hub and forming a flange, the flange facilitating placement of the needle assembly in the fully closed position through the application of pressure to the flange.

12. The needleshield assembly of claim 11, wherein the coupling member comprises a proximal end having a lower surface and wherein the cover comprises an upper surface adjacent the hinge, wherein the lower surface of the coupling member is adapted to contact the upper surface of the cover when the needle assembly is rotated into the fully closed configuration.

13. The needleshield assembly of claim 12, wherein the lower surface of the coupling member and the upper surface of the cover are substantially planar.

14. A method of dispensing or withdrawing a fluid with a needle and preventing re-use of the needle, comprising:
providing the needleshield assembly of claim 1 in an open configuration in which the needle and needle hub are outside the cover;
attaching the needle hub to a syringe;
dispensing or withdrawing the fluid through the needle and needle hub; and
rotating the needleshield assembly around the hinge from the open configuration to a fully closed configuration in which the needle and needle hub are retained within the cover and the locking protrusion of the needle hub is received within the lock aperture of the cover, wherein the inwardly projecting protrusion is simultaneously brought into contact with the outer surface of the needle hub so as to pierce the surface of the needle hub when the needle assembly is rotated into the fully closed configuration, the needle assembly thereby being secured within the cover and rendered incapable of reuse when placed in the fully closed configuration.

15. The method of claim 14, wherein the cover further comprises a lever protruding from an exterior surface of the proximal end of the cover, further comprising the step of applying pressure to the lever in order place the needleshield assembly in the fully closed configuration.

16. A method of dispensing or withdrawing a fluid with a needle and preventing re-use of the needle, comprising:
providing the needleshield assembly of claim 3 in a closed, ready-to-use configuration, wherein the retaining pin is retained within the lock aperture of the cover and the needle and needle hub are retained within elongate body of the cover;
rotating the needleshield assembly around the hinge from the closed, ready-to-use configuration to an open configuration in which the needle and needle hub are outside the cover;
attaching the needle hub to a syringe;
dispensing or withdrawing the fluid through the needle and needle hub; and
rotating the needleshield assembly around the hinge from the open configuration to a fully closed configuration in which the needle and needle hub are retained within the cover and the locking protrusion of the needle hub is received within the lock aperture of the cover, wherein the inwardly projecting protrusion is simultaneously brought into contact with the outer surface of the needle hub so as to pierce the surface of the needle hub when the needle assembly is rotated into the fully closed configuration, the needle assembly thereby being secured within the cover and rendered incapable of reuse when placed in the fully closed configuration.

17. The method of claim 16, wherein the cover further comprises a lever protruding from an exterior surface of the proximal end of the cover, further comprising the step of applying pressure to the lever in order place the needleshield assembly in the fully closed configuration.
